(19) United States
(12) Patent Application Publication
(10) Pub. No.: US 2009/0186332 A1
(43) Pub. Date: Jul. 23, 2009

(54) PREPARATION AND STORAGE OF STABLE, ANTIMICROBIALLY ACTIVE MATERIALS

(76) Inventors: Ernest K. Manders, Pittsburgh, PA (US); Christan D. Manders, Pittsburgh, PA (US)

Correspondence Address: THE SONI LAW FIRM, 35 North Lake Ave., Suite 720, PASADENA, CA, 91101, US

(21) Appl. No.: 12/293,079

(22) PCT Filed: Mar. 15, 2007
(86) PCT No.: PCT/US07/06591

§ 371 Date: Dec. 29, 2008

Related U.S. Application Data
(60) Provisional application No. 60/782,395, filed on Mar. 15, 2006.

(51) Int. Cl. A01N 1/02; A61L 2/00

(52) U.S. Cl. 435/1.1; 422/22

(57) ABSTRACT

A method for the preparation of antimicrobially active materials is presented. The invention involves taking a base material such as allografts, xenografts, polymers, metals, and ceramics and combining it with an antimicrobially active agent, such as antibiotics, antibacterials, antifungals, antivirals, disinfectants, and polypeptides, after which it is irradiated with ionizing radiation to sterilize and stabilize the combined material. The resulting antimicrobially active material may then be stored at ambient temperature while maintaining its antimicrobial activity and the structural integrity of the base material. The invention is particularly useful for both preventing and treating a variety of infections and for increased safety in reconstructive procedures.

### CROSS-REFERENCES TO RELATED APPLICATION

This application claims the benefit of U.S. Provisional Application Ser. No. 60/782,395, filed Mar. 15, 2006, the content of which is hereby incorporated herein by reference.

### BACKGROUND OF THE INVENTION

The present invention relates to a method to prepare human allografts, xenografts derived from mammals, reptiles, birds, amphibians, fish, and invertebrates, both naturally occurring and synthetic polymeric materials, metals, and ceramics for use as antimicrobially active materials. This invention describes the preparation of a human allograft, xenograft, natural and synthetic polymeric materials, metals, and ceramics with the addition of a compound with antimicrobial activity antimicrobially bound to the base material that is then irradiated with ionizing radiation so as to sterilize and stabilize the combined material. These combined materials are able to be stored at ambient temperature and to elicit biological responses in the person or animal, or industrial process into which or onto which the combined material is placed. The goal of such a combination is the suppression of local bacterial or fungal growth under a surface dressing or in areas surrounding an implant. The present invention is further directed at creating an antimicrobially active material configured with antimicrobials that may be conventional antibiotic drugs such as, but not limited to, penicillin, gentamicin, and kanamycin; disinfectants such as, but not limited to, silver ion, hexachlorophene, and povodine iodine; antifungals such as, but not limited to, polyene antimycotics, imidazol and triazole, and allylamines; antivirals such as, but not limited to, amantadine, rimantidine, pleconaril, acyclovir, and lamivudine; viricides; antiparasitics such as, but not limited to, antinematodes, anticestodes, antiamoebics, antiprotozoals; as well as polypeptide agents such as, but not limited to, maganins and agents that form pores in the bacterial cell wall, bound to a base material such that the antimicrobial agent adheres to, coats, or is embedded within the base material. The contents of U.S. Pat. Nos. 5,534,026 and 5,697,383 and U.S. Publ. No. 2005/0043,235 are each hereby incorporated herein by reference for all purposes.

By way of background, allograft skin has been shown to provide an excellent temporary skin coverage for burn patients, acting as a biological dressing. Allograft skin protects the wound from desiccation, contamination, and decreases wound pain. When allograft skin shows general adherence to a burn wound and evidence of graft vascularization within 48 to 72 hours of application, one can anticipate an excellent take of autograft skin applied to the wound following removal of the allograft skin. Limitations of fresh allograft skin includes the dearth of material, the need for refrigerated storage facilities, and a limited “effective” shelf life of approximately seven to ten days when the tissue is stored at 4 degrees Celsius. The possibility of disease transmission requires careful donor selection [Pruitt, B A et al., Arch. Surg. 119, 312 322, (1984)]. Other allograft materials such as bone and soft tissues face similar storage limitations.

Current developments in the field of allograft skin products focus on culturing epidermal cells to form skin like coverings to be used as skin allografts as referenced in U.S. Pat. No. 5,015,584. Cryopreservation of allograft is commonly used, which retains the viability of the donor cells to some extent. It was previously believed that living cells were required for the success of skin allograft. However, good results have been obtained using methods which preserve the allograft without retaining the viability of the cells, such as preservation with glycerol [Kreis R W, et al., J Trauma 29(1), 51 54 (1989)] [Hermans, M H E, Burns 15(1), 57 59 (1989)], silicone fluid [Ballantyne, D L Jr. et al., Cryobiology 8, 211 215, (1971)] or lyophilization [Young, J M et al., Arch. Surg. 80(Feb.), 208 213, (1960)].

Fresh frozen allograft skin and lyophilized allograft skin have limitations such as demanding processing procedures. The requirements for such procedures confine the preparation of either material to special centers having proper facilities. The lyophilized material has an essentially unlimited nonrefrigerated shelf life, while the frozen material has a similarly prolonged shelf life provided proper refrigeration is maintained. Either material can be easily and rapidly prepared for use by rehydration or thawing. Lyophilized allograft skin generally adheres less well to the wound and is less able to reduce the bacterial count on the wound surface than fresh allograft skin [Pruitt, B A et al., Arch. Surg. 119, 312 322, (1984)].

U.S. Pat. Nos. 3,645,849 and 3,743,480 describe processes for sterilization of biological material (e.g., blood serum) by microwave irradiation. Methods for preparing and sterilizing biological tissues such as heart valves, veins, cartilage, ligaments and organs for use as bioprostheses are described in U.S. Pat. No. 4,994,237. The source of irradiation is a microwave oven. This method tends to heat the specimen and destroy its structure. A method of sterilization of biological material by ultraviolet light is described in U.S. Pat. No. 4,880,512. Ultraviolet light is an efficient method of sterilization but it does not penetrate through objects such as skin very well. Consequently, this method is not always secure. In addition, Ultraviolet Light is not efficient for batch sterilization.

Another widely used method of biological tissue preservation and sterilization, which does not retain cell viability, is gamma irradiation. This method has been used extensively in the preservation of bone allograft, with good results. It has also been used in the preservation of donor cartilage [Dingman R O et al., Plast. Reconstr. Surg. 28(5), 562 567, (1961)], blood vessels, heart valves [Wright K A et al., Sterilization and Preservation of Biological Tissues by Ionizing Radiation. Vienna, International Atomic Energy Agency, 107 118, (1970)], dura mater, and sclera [Colvard D M et al., Am. J. Ophthal., 87(4), 494 496, (1979)]. Irradiation sterilization of the tissue permits storage at room temperature, a considerable advantage when low temperature storage is unavailable. U.S. Pat. No. 4,351,091 employs gamma and x ray irradiation to preserve a corpse to kill bacteria and other microorganisms that contribute to the decomposition of a corpse. This patent does not address infectious diseases such as viruses or the feasibility of preparing or preserving the corpse for organ donation.

With the use of allograft skin, there is an associated risk of the transmission of disease, including the human immunodeficiency virus (HIV). Skin banks around the world were virtually closed down for two or more years after the reported transmission of HIV from allograft skin [Clarke J A, Lancet 1,983, (1987)]. Gamma irradiation at ranges of 250,000 cGy to 2.5 million cGy has been shown to inactivate HIV [Hiemstra H et al., Transfusion, 31(1), 32 39, (1991)] [Spire B et al., Lancet, 1, 188 189, (1985)]. The effect of gamma irradiation on human coagulation factors found in human plasma and on virus suspended in plasma or other types of suspending medium has been studied [Kitchen, A D et al., Vox Sang 56, 223 229, (1989)].

The base materials including allografts, xenografts, polymeric materials, metals, and ceramics often lack antimicrobially active agents that may be lost due to processing or are not naturally occurring on the base material. The present invention enhances the base materials such as allografts, xenografts, polymeric materials, metals, and ceramics for implant or surface usage by adding antimicrobially active agents to them. The base material may be in the solid, liquid, or aerosol state. The addition of these antimicrobial elements can greatly increase the functionality of the combined material when used in or on the body or in some cases when used in industrial processes for catalysis, fermentation, and other reactions. The antimicrobial properties of the material will allow it to decrease bioburden on a wound or in the body of a human or animal. Implanted materials can often cause infection within the body and the creation of a material that has antimicrobial properties will prevent many potential infections.

What is needed and heretofore unavailable is the creation of an antimicrobially active material that combines a base substrate material with the addition of antimicrobially active agents which may not be naturally occurring on the base material or may not be present in the desired concentrations. This allows for creating custom-made antimicrobially active materials to better achieve prescribed effects. These antimicrobially active materials will also be stable and storable at ambient temperature for a sustained period of time.

### SUMMARY OF THE INVENTION

In accordance with the present invention, a new method for the preparation, stabilization, and sterilization of antimicrobially active materials is presented. This invention describes the preparation of a human allograft (including but not limited to skin, bone, tendon, fascia, cartilage, nerves, vessels, valves, corneas, organs, and component tissues of organs), xenograft (including but not limited to skin, bone, tendon, fascia, cartilage, nerves, vessels, valves, corneas, organs, and component tissues of organs), a natural or synthetic polymer, metals, and/or ceramics that includes the addition of antimicrobially active agents including but not limited to: antibiotics, antifungals, antivirals, disinfectants, and polypeptide agents bound to the material. This antimicrobially active material when introduced into or onto the body can affect the body in a desired way (including, but not limited to accelerating, inhibiting, or maintaining in an unaltered state, healing, vascularization, fibrosis, cell proliferation, cell death, and/or an immunologic response). The addition of the antibiotic, antifungal, antiviral, disinfectant, and polypeptide agents(henceforth “antimicrobially active agents”) will be capable of destroying bacteria, fungi, and viruses and the combined material will be storable at ambient temperature following processing, and may be or may not be sterile. The present invention is a combination of these two elements, (a) an antimicrobially active agents or drugs and, (b) a base material formed from allograft, xenograft, natural or synthetically derived polymeric materials, metals, and/or ceramics that will be stable at ambient temperature following irradiation and that will produce an antimicrobially active material that would elicit an antimicrobial 1 response in treating a person or animal or as an industrial tool. This is an improvement on the prior art, which does not allow for sustained storage and stability of materials that include antimicrobially active agents, in particular antibiotics, antifungals, antivirals, and antiparasitics at ambient temperature. In addition, the present invention provides for a sterile and stable allograft, xenograft, polymeric material, metal, and/or ceramic and the attachment of the antimicrobially active agent to the base material prior to or following irradiation.

The method and products of the present invention have applications in many areas. In the case of skin, such applications include, but are not limited to, wound and bum therapy, venous stasis ulcers, diabetic foot ulcers, full thickness ulcers, Mohs surgery sites, skin graft donor sites, partial thickness wounds, areas of dermabrasion, temporary coverage of exposed abdominal viscera including small bowel and liver, exposed pericranium and cranium, fasciotomy sites, as a “Canary Test” on a wound bed before autografting, and areas of excision which are not closed pending final pathology report. The allograft or xenograft skin may be combined with an antimicrobially active agent that reduces bioburden and can increase healing rates while reducing infection. For instance, the antimicrobial may be combined with allograft, xenograft, polymeric materials, metals, or ceramics and irradiated to allow the combined material to be stable at ambient or room temperature. The could help cell proliferation to close the wound while the allograft, xenograft, or other material would provide an occlusive wound covering that would create a wound healing environment and would prevent the wound from drying out.

In the case of musculoskeletal allografts or xenografts, such applications could include bone grafts including but not limited to osteochondral grafts and chondral grafts, tendon grafts, nerve grafts, cartilage grafts, etc. These grafts may be coated, embedded, or bound with an antimicrobially active element that will create an action when used on a patient. Bone grafts could be implanted with antimicrobial agents to prevent infection at the implant site.

In the case of natural or synthetic polymeric materials, metals, and ceramics the material could be used as an implantable material or as a surface covering. The polymeric material could be constructed in various shapes, forms, and consistencies to create the desired material properties for each individual application. Polymers from biological sources that can be utilized include, but are not limited to: Polygalacturonic acid, Hydroxypropyl cellulose, Hydroxyethyl cellulose, Heparin, Collagen, Gelatin, Carboxymethyl cellulose, Pectin, Algin, Ethyl cellulose, Glycosaminoglycan, Chitin/Chitosan, and other polysaccharides. Suitable metals for use as a base material for the present invention include, but are not limited to, medical grade stainless steel, titanium, chrome vanadium steel, silver, platinum, gold, and nickel-titanium alloys, such as nitinol. Suitable ceramics for use as a base material for the present invention include, but are not limited to, alumina, zirconia, silicon nitride, silicon carbide, steatite and cordierite.

The antimicrobially active material could also be used in industrial or manufacturing processes. The antimicrobially active material could be an agent used to initiate chemical or biological processes or to catalyze materials. These antimicrobially active materials could be used to better stabilize starch processing enzymes or proteases that are used in detergents. These materials could be altered to increase the temperature stability of the enzymes.

Ionizing radiation, such as Gamma Irradiation from a Cobalt 60 source, has been earlier shown to inactivate HIV and has been used previously to sterilize allografts of bone and other tissues, but has not previously been used to sterilize, stabilize, and preserve antimicrobially active materials comprised of the combination of antimicrobially active agents and base materials. Human allografts were irradiated in the present invention and applied as a temporary wound dressing on a skin graft donor site. When compared with a frozen skin allograft on the same recipient, the irradiated allograft proved to be as effective. It offers the potential of a low cost, safe and effective treatment that can be used widely and without extensive training or extensive facilities.

An object of this invention is to develop a method of sterilizing and storing a antimicrobially active material so that the risk of transmission of infectious diseases, particularly bacterial, fungal, and viral diseases, is eliminated or significantly reduced. An additional object of this invention is to provide a method of preparing an antimicrobially active material that is inexpensive and includes additional antimicrobially active agents to enhance the base material's functionality in the patient and easily available to a large percentage of the medical community. Another object of this invention is to allow for the preservation of the antimicrobially active materials without the need for refrigeration or other treatment which would result in additional expense.

Other features and advantages of the invention will become apparent from the following detailed description, which illustrates, by way of example, the features of the invention.

### DETAILED DESCRIPTION OF THE INVENTION

The present invention is directed to the use of ionizing irradiation (for example, gamma irradiation) to sterilize and prepare allografts from humans, xenografts, synthetic or naturally occurring polymers, metals, and ceramics that include the addition of antimicrobially active agents such as antibiotics, antifungals, antivirals, and antiparastics for use as an antimicrobially active material. Because of the risk of the transmission of infectious diseases such as HIV, hepatitis, and other bacterial, fungal, and viral diseases, the use of a safe, effective and inexpensive method of preparing an antimicrobially active material has become apparent. There is also a need for shelf-stable antimicrobially active materials to treat disease or for industrial applications. This invention describes the preparation of an irradiated material that includes antimicrobially active agents such as antibiotics, antifungals, antivirals, and antiparastic, and/or drug entities bound, attached, embedded to the material to elicit a biological response in the body. The addition of the antimicrobially active agents will create a material that can elicit a specified response in the body and reduce potentially harmful microbes.

Donor skin from an HIV and hepatitis negative donor was obtained from the skin removed during a thighplasty. This skin was harvested using a power dermatome and sheets of skin 0.014 in. thick were obtained. These were placed immediately in Tis-u-Sol (Baxter; Deerfield, Ill.), a balanced salt solution, and stored overnight at 4 degrees Celsius. The harvested skin was then rinsed three times in Tis-u-Sol, and divided into several groups. One sample was placed in a solution of Eagles Minimal Essential Medium and dimethyl sulfoxide (DMSO) and frozen in liquid nitrogen. One piece was placed directly in formalin, to serve as a control for histological studies. Other pieces were placed in Tis-u-Sol in glass or plastic containers for irradiation with 3.0 million cGy at 23 degrees Celsius using a Cobalt 60 source. The allograft skin may be placed in a wide variety of solutions including but not limited to: glycerol, balanced salt solutions, Wisconsin's solution, etc.

The present invention can be practiced by irradiating the material substrate and the added antimicrobially active element for a period of time sufficient to provide a sterilizing and/or preserving dose of ionizing radiation, such as gamma radiation from a Cobalt 60 source. Accordingly, such dosage is calculated using ordinary and usual parameters (i.e., medium size, etc.) of dosimetry. Irradiation dosages, sufficient to effect sterilization, are known in the art. Other irradiation variables such as oxygen content, humidity, temperature, time, dose rate, can be altered so as to achieve the optimum dose. One of normal skill in the art will be capable of altering these variables so as to achieve a suitable result. Rinsing is not obligatory to practice the invention. As additional controls, several pieces of skin were left in Tis-u-Sol at 23 degrees Celsius both with and without antibiotics (5000 U/cc penicillin and 5000 mcg/cc streptomycin) for the amount of time required to irradiate the 3 million cGy samples. At the end of the irradiation period, a sample of the irradiated skin and a sample of each of the 23 degrees Celsius controls were cultured and placed in formalin for analysis. The remainder of the irradiated skin was stored at 23 degrees Celsius (room temperature) in the closed containers employed for the sterilization procedure and may be stored for an extended period of time.

It is contemplated by the present invention that the irradiated antimicrobially active material made according to teachings of the present invention may be stored at ambient or room temperature for one day, two days, three days, five days, seven days, ten days, twenty days, thirty days, sixty days, one hundred eighty days, three hundred sixty five days, two years, and even longer. The storage time at ambient temperature will be dependent on the individual antimicrobially active agents and the type of base material(s) used. The finished antimicrobically active material will be shelf-stable, storable at ambient temperatures and the antimicrobial activity will be stabilized such that the structural integrity of the base material will be maintained with an enhanced antimicrobial activity after processing.

After 14 days, a sample of cryopreserved skin and two samples of the 3 million cGy irradiated skin were placed on a thigh skin graft donor site of a healthy volunteer. A portion of each allograft was placed in formalin for analysis at the time, and 2 mm punch biopsies were obtained at 3, 6, 8, 10, 13, 17, and 24 days post op. All samples were stained using hematoxylin and eosin, as well as colloidal iron, and all histological samples were numbered and evaluated in a blinded fashion.

Cultures were negative for bacteria for both the control samples and the irradiated samples

Throughout the study, the patient reported minimal pain from all areas of his donor site; no evidence of infection was seen at any time.

The clinical course of the allografts showed that at postoperative day two, both grafts looked somewhat pink and were firmly adherent to the graft bed. At day three both grafts were still pink and intact, but some epidermolysis was visible on the frozen allograft. By postoperative day six, the superficial epidermis of the frozen allograft had almost completely sloughed, while in contrast the irradiated allograft remained intact and supple. Histological examination at this point shows the frozen allograft dermis overlying the patient's own epidermis and dermis, while the irradiated graft appears intact but with nonviable cells. Between postoperative day eight and thirteen, the frozen allograft began to develop some areas of epithelialization over the remaining allograft dermis, while the irradiated allograft began to form a thin eschar interspersed with some areas of epithelialization. By postoperative day seventeen the frozen allograft began to slough completely, while the site of the irradiated allograft was predominantly epithelialized, with some areas of eschar still remaining. Histologic examination shows the frozen allograft to be well epithelialized over the allograft dermis, with the patient's dermis and epidermis underneath; while the nonviable cells of the irradiated graft have been replaced with living cells. At postoperative day 27 the frozen allograft site still had many areas lacking epithelialization due to islands of retained allograft dermis, while the irradiated site was predominantly epithelialized.

We have shown that irradiated allograft is as effective a biological dressing as conventional frozen allograft. HIV and other viruses are inactivated by the radiation dose used in the present invention.

The results in this patient indicate that the cryopreserved allograft does indeed survive to form a viable skin layer over the patient's own tissue until it is rejected. The irradiated allograft forms an inert, protective barrier which sloughs after regrowth of the patient's own epidermis. Both forms of allograft performed well as a dressing, providing good coverage and pain relief as well as protection from infection. The irradiated allograft, however, produced a stable epithelial surface ten days before the cryopreserved allograft.

Skin allograft preservation by ionizing irradiation (for example, gamma irradiation) has many advantages, and makes skin allograft use a possibility in areas where it is not currently available, such as small hospitals, doctors' offices, and developing countries of the world. The preparation of irradiated skin allograft is inexpensive and simple to perform, requiring only basic materials and access to a source of ionizing radiation, such as Cobalt 60. Irradiated allograft can be stored on the shelf at room temperature and does not require liquid nitrogen or low temperature freezer storage. Application of irradiated skin requires no thawing, washing or rehydration, as found with other methods of skin preservation.

The only factors limiting the usefulness of this technique are the availability of cadaveric skin and a source of ionizing radiation, such as Cobalt 60. The low cost of the method and the fact that the skin is virus free, and specifically HIV free, will make this a most attractive method of preparing allograft skin for patients with burns and other wounds.

The present invention includes a method for the addition of antimicrobially active agents such as antibiotics, antifungals, antivirals, and antiparasitics that favor wound vascularization and healing to a human skin allograft that can be irradiated (for example, terminal sterilization) and stored at room temperature. The method and product of the present invention combines these two elements, (a) an antimicrobially active agent or agents such as antibiotics, antifungals, antivirals, and antiparasitics and, (b) a base material such as allograft, xenograft, polymeric materials, metals, or ceramics both of which are room temperature stable after irradiation to provide an antimicrobially active material to elicit a response in or on the body. The combination of a base material and antimicrobially active agents provides a novel room temperature-stable preparation of an antimicrobially active material. Heretofore, it was not understood that these entities could be combined, irradiated, stabilized, and stored at room temperature. Accordingly, it has been generally accepted that antimicrobially active agents must be stored in the cold until used. The application of materials with antimicrobially active agents incorporated into them provides a mechanism of delivering antimicrobial agents to wounds at biological temperatures. This invention therefore also provides the preparation and delivery mechanism of antimicrobially active agents heretofore not available.

The methods and products of the present invention allow the simultaneous delivery of antimicrobially active agents to wounds while providing an ideal closure for healing. The present invention could involve skin with the epidermal layer or only the dermal layer of the skin. This could prove an advantage for wounds that lack adequate vascularity or whose environment has diminished the supply of the usual factors present in a normally healing wound. The invention would uniquely provide an adherent wound closure and thereby an ideal healing environment, and at the same time it would also allow the ready delivery of antimicrobial agents that could eliminate harmful microbes that could interfere with healing or the health of the patient. As will be appreciated by those of ordinary skill in the art, various methods, procedures and systems are available. The binding or attachment elements of the invention are subsequently described. The antimicrobially active agents may be combined with the human allograft, xenograft, natural or synthetic polymeric material, metal, or ceramic by one or more, but not limited to of the following methods available for providing a mechanism of addition and binding of the antimicrobial agents to the allograft.

The binding or attachment elements of the invention are subsequently described. The antimicrobially active agents may be combined with the human allograft, xenograft, natural or synthetic polymeric material, metal, or ceramic by one or more, but not limited to of the following methods:

Combination of Base Material with Biologically Active Agent:

The combination of the base material and the antimicrobially active agents such as antibiotics, antifungals, antivirals, disinfectants, and peptides may be made in several ways. Three such methods, which are not meant to be the only methods available, include simple adsorption, covalent bonding such as with formation of urethane bonds, and sequestration with formation of salts. Additionally, antimicrobial active agents may be injected, inserted, or embedded into the base material.

Simple Adsorption:

The base material may be combined with antimicrobially active agents by the act of simple immersion of the base material in a solution containing a suitable concentration of the antimicrobially active agent(s) of interest. Such immersion may be conducted at temperatures from 0° to 40° C. for intervals of several seconds to hours and even days.

The antimicrobially active agents are bound by hydrogen bonding and ionic interactions and are therefore readily available for release in a therapeutic environment. The antimicrobially active agents typically have charged groups like —N⁺H₃ and —CO₂⁻, and groups that are highly polar, such as —OH and —SH. Similar groups are found on allograft and xenograft materials and many natural and synthetic polymers, metals, and ceramics allowing binding interactions to occur with resultant immobilization of the desired antimicrobially active agents on the base material of interest.

Covalent Bonding:

Antimicrobials commonly contain amine groups (—NH₂), sulfhydryl groups (—SH), carbonyl groups (—CO₂), and oxygen species (—O). Polyisocyanate species may react with acidic groups in the following way:

O═C═N—R—N═C═O+—XH

where X═—NH₂, —SH, —CO₂, —O. A preferred cross-linking agent is the polyether polyisocyanate sold as Hypol© Foamable Hydrophilic Prepolymer (W. R. Grace & Co., Lexington, Ma.). This produces a reaction:

RNCO+H2O→RNHCOOH

(Unstable carbamic acid)

RNHCOOH→RNH2+CO2↑

(Amine formation and gas generation)

RNH2+RNCO→RNHCONHR

(Urea chain extension Cross-linking formation)

Other cross-linking agents may be suitable such as alkylene polyacrylates, alkylene polymethacrylates, alkylene glycolpolymethacrylates, polyaldehydes and other cross-linking reagents that will cross-link molecules with reactive protic groups. Suitable initiators of polymerization may be required, including as examples but not limited to azobisisobutylnitrile, peroxide initiators such as benzoyl peroxide, isopropyl peroxide and similar reagents. Such cross-linking will result in a covalent bond between the allograft, xenograft, polymeric material, metal, or ceramic and the chosen antimicrobial agent.

Salt Formation:

Antimicrobials may be precipitated and bound by alkali metal phosphates. Calcium phosphate as hydroxyapetite is an example of a polymer capable of binding molecules to surfaces. This agent is utilized to bind a drug preventing fibrosis to drug eluting stents.

The antimicrobially active material can be loaded with the desired antimicrobially active agent(s), which is believed to occur by ionic binding involving ionic sites on the biopolymer, with the desired bioactive agent, which may be antimicrobial drugs or macromolecules such as, antibacterial agents, antibacterial agents, e.g., sulfonamides such as sulfadiazine, sulfamerazine, sulfamethazine, sulfisoxazole, and the like, antimalarials such as chloroquine and the like, antibiotics such as the tetracyclines, nystatin, streptomycin, cephradine and other cephalosporins, penicillin, semi-synthetic penicillins, griseofulvin and the like, These substances are frequently employed either as the free compound or in a salt form, e.g., acid addition salts, basic salts like alkali metal salts, etc. Other therapeutic agents having the same or different physiological activity can also be employed in the pharmaceutical preparations within the scope of the present invention. Typically, the bioactive agent dissolved in a suitable solvent will be contacted with the starting material by immersion. The loading of the base material may be readily determined based upon the uptake of the starting material (allograft, xenograft, polymer, metal, or ceramic) of the antimicrobial agent.

The following are examples, which are illustrative and not intended to be limiting, antimicrobial agents that could conceivably be combined with an allograft of the present invention for therapeutic benefit:

The base material can be loaded with the desired antimicrobial agent(s), which is believed to occur by ionic binding involving ionic sites on the base material, with the desired bioactive agent, which may be antimicrobial drugs or macromolecules such antimicrobial agents. The following are examples, which are illustrative and not intended to be limiting, of antimicrobials that could conceivably be combined with an allograft of the present invention for therapeutic benefit: Penicillins, Cephalosporins, Carbapenems, Monobactams, Aminoglycosides, Tetracyclines, Macrolides, Sulfonamides, Fluoroquinolones, Streptogramins, Oxazolidinones, Lincosamines; miscellaneous agents (such as, but not limited to, Vancomycin, Metronidazole, Clindamycin, Spectinomycin, Chloramphenicol, and Tremethoprim); antifungal drugs (such as, but not limited to, Amphotericin B, Flucytosine, Itraconazole, Fluconazole, Ketoconazole, Miconazole, Nystatin); and antibacterial agents commonly used as urinary tract disinfectants (such as, but not limited to, Fosfomycin, Methenamine mandelate, Methenamine hippurate, Nalidixic acid, and Nitrofurantoin).

### FIRST EXAMPLE

### Allograft Skin

Allograft skin may be combined with silver ion and then packaged and irradiated with production of a sterile allograft storable at ambient temperature and possessing an enhanced ability to nourish the growth of new vessels in a wound to which it is applied. This is accomplished by rinsing recovered allograft skin to wash off any antibiotics and freezing medium that may be present. One then places the allograft dermis-side down on a piece of Telfa pad saturated with a solution of silver ion from silver nitrate, for example, at a concentration of 0.5 to 3% (w/v) in a balanced salt solution or other liquid media. (Higher concentrations of 4 and 5% may actually injure the wound.) The skin is allowed to absorb the silver ion solution for 15 minutes at room temperature. The skin is then packaged in a moist dressing and sealed in a packaged made of a composite of plastic and foil. This is sealed and then irradiated with at least 30 kGy of ionizing radiation. After this last step, the skin can be stored at ambient temperature.

### SECOND EXAMPLE

### Allograft Bone

Allograft bone is commonly used to aid in the reconstruction of fractures and in the successful fusion of a patient's bone, The bone graft is often placed in an area of injury or other compromise, such as a site of a failed fusion. The fact of traumatic injury and previous surgery all raise the risk of infection for this follow-on surgery. The graft is not vascular and faces a real risk of infection. This risk may be reduced by combining the graft with an antimicrobial such as silver ion.

For this embodiment small pieces of allograft bone from 1 to 5 mm in diameter are simply immersed in a solution of silver ion with a concentration of 0.5 to 3% in a balanced salt solution. The fragments are then lifted from the solution and allowed to drain until moist but no longer dripping. The treated bone allograft is then placed in a suitable container and sealed in an impervious container which may be a bottle or a bag. The container is then subjected to 30 kGy of ionizing radiation after which the allograft and the adsorbed silver ion are stable at room temperature for an extended period of time.

### THIRD EXAMPLE

### Pollulan Polymer as VEGF Carrier

Pollulan is a biological biodegradable polymer that may be formed into a wafer which can serve as a delivery vehicle. In this application a wafer of the polymer of size chosen is immersed in a solution of silver ion with a concentration of 0.5 to 3% in a balanced salt solution for 15 minutes at room temperature. The wafer is then lifted from the bath and allowed to drain and then covered with a plastic sheet which is then placed in a sealable container. The polymer carrier and its silver ion cargo are then irradiated with at least 30 kGy of ionizing radiation. Thereafter the package can be stored for extended periods of time at ambient temperature.

### Radiation:

Ionizing radiation may be administered by a source such as a commercial Cobalt 60 or electron beam source. The dose may be selected according to the needs of the material at hand. Bacterial sterilization may be accomplished with reference to tables of radiation sensitivity of bacteria and the need to reduce the bacterial count to less than 10-6 colony forming units. The bioburden present at the start is important for this calculation as is familiar to anyone skilled in the art of radiation sterilization. Biological samples may be sterilized of viruses if an adequate dose of radiation is selected. The common pathogens screened for in donor selection are eliminated by a cumulative dose of 30 kGy or more. Thus, high dose ionizing radiation is capable of sterilizing biological specimens and thereby may eliminate the risk of inadvertent infection by transplantation of allograft and xenograft materials. Appropriate doses may vary according to the needs of a particular situation, varying from 2000 cGy to over 50 kGy, with the most frequent dose being between 3 and 35 kGy.

Radiation may be administered at temperatures from the very cold (liquid nitrogen and dry ice) to room temperature and above. Rates of radiation delivery may vary from about 0.5 kGy/hr to about 4.0 kGy/min for a period of about 5 minutes to about 40 hours. Low temperature renders radiation less effective in inactivating bacteria and viruses. Someone skilled in the art of radiation sterilization knows how to adjust the dose administered to account for the potentially protective effects of low temperature.

Biological materials subjected to high dose irradiation may be stored at room temperature. The storage temperature includes temperatures from 0° to 40° C. The duration of storage may vary from 5 minutes, to 15 minutes, to 1 hour, to 12 hours, to 1 day, to 7 days, to 30 days, to six months, to 1 year, to 2 years, to 6 years and beyond, and intermediate times in between.

### Methods of Use:

Surface Application:

Both acute and chronic wounds may benefit from antimicrobial agents delivered in pharmacologic doses. As an example of this allograft skin delivering ionic silver would promote healing in chronic wounds by reducing significantly the level of bacterial colonization. Allograft would offer the additional advantages of closing the wound to bacteria invasion and preventing desiccation.

Implantation:

Musculoskeletal tissues are typically implanted in the body in an attempt to reconstruct or repair damaged elements of the musculoskeletal system. An example of an antimicrobially active material that could enjoy widespread use is bone allograft bearing silver ion. Such allograft would be less likely to become infected at sites of trauma surgery, re-operation, and in very large wounds at risk of contamination.

Industrial Use:

Industry makes widespread use of enzymes and fermentation. Fermentation in particular could be aided by the addition of a natural polymer such as a polysaccharide with embedded enzyme that would help hydrolyze the polysaccharide to present its constituent sugars as a substrate for fermentation. Prepared as described in this disclosure such a functional substrate could include an antibacterial that would prevent contamination of fermentation by unwanted bacterial growth.

### Veterinary Use:

Large animal veterinarians often must treat their animal patients with many of the technologies that are available to human patients. A fracture in a race horse's leg could be addressed with allograft bone enhanced by addition of antimicrobial silver ion for prevention of infection in an animal with limited means for hygiene. This would favor recovery and the preservation of a potentially very valuable animal for breeding, personal companionship, and possibly even resumption of racing.

While particular forms of the invention have been illustrated and described, it will also be apparent to those skilled in the art that various modifications can be made without departing from the inventive concept. References to use of the invention with a specific compound, chemical or radiation source and with respect to a particular disease or condition are by way of example only, and the described embodiments are to be considered in all respects only as illustrative and not restrictive. The present invention may be embodied in other specific forms without departing from its spirit or essential characteristics. Accordingly, it is not intended that the invention be limited except by the appended claims.

We claim:

1. A method of preparing an antimicrobial material, comprising the steps of:
providing a base material;
providing an antimicrobial agent;
combining the antimicrobial agent with the base material so as to form an antimicrobial material; and
exposing the antimicrobial material to a source of ionizing radiation sufficient to sterilize and stabilize the antimicrobial material.

2. The method of claim 1, wherein combining the antimicrobial agent to the base material includes using an adsorption process.

3. The method of claim 1, wherein combining the biologically active agent to the base material includes using an absorption process.

4. The method of claim 1, wherein combining the biologically active agent to the base material includes using a covalent bonding process.

5. The method of claim 1, wherein combining the biologically active agent to the substrate includes sequestration with salt formation.

6. The method of claim 1, further including storing the biologically active material at a temperature above freezing without substantial degradation of the base material or the antimicrobial agent, while maintaining sterility and stability of the antimicrobial material.

7. The method of claim 6, wherein storing the biologically active material is performed at ambient temperature for a period of at least one day.

8. The method of claim 1, wherein providing a base material includes using an allograft.

9. The method of claim 8, wherein using an allograft includes providing a material selected from the group consisting of skin, bone, tendon, fascia, cartilage, nerves, vessels, valves, corneas, organs, and component tissues of organs.

10. The method of claim 1, wherein providing a base material includes using a xenograft.

11. The method of claim 10, wherein using an xenograft includes providing a material selected from the group consisting of skin, bone, tendon, fascia, cartilage, nerves, vessels, valves, corneas, organs, and component tissues of organs.

12. The method of claim 1, wherein providing a base material includes using a polymer.

13. The method of claim 12, wherein using a polymer includes providing a material selected from the group consisting of Polygalacturonic acid, Hydroxypropyl cellulose, Hydroxyethyl cellulose, Heparin, Collagen, Gelatin, Carboxymethyl cellulose, Pectin, Algin, Ethyl cellulose, Glycosaminoglycan, Chitin/Chitosan, and polysaccharides.

14. The method of claim 1, wherein providing a base material includes using a metal.

15. The method of claim 14, wherein using a metal includes providing a material selected from the group consisting of medical grade stainless steel, titanium, chrome vanadium steel, silver, platinum, gold, and nickel-titanium alloys, such as nitinol.

16. The method of claim 1, wherein providing a base material includes using a ceramic.

17. The method of claim 16, wherein using a ceramic includes providing a material selected from the group consisting of alumina, zirconia, silicon nitride, silicon carbide, steatite and cordierite.

18. The method of claim 1, wherein providing an antimicrobial agent includes using a material that reduces the bioburden in vivo.

19. The method of claim 1, wherein providing a biologically active agent includes using a material selected from the group consisting of an antibiotic drug, a disinfectant, and a polypeptide.

20-22. (canceled)

23. An antimicrobial material prepared according to claim 1.

24. (canceled)

25. A antimicrobial material, comprising:
a base material; and
an antimicrobial agent,
wherein the antimicrobial agent is combined with the base material so as to form an antimicrobial material, and
wherein the antimicrobial material is exposed to a source of ionizing radiation sufficient to sterilize and stabilize the antimicrobial material.
